(19) United States
(12) Patent Application Publication
(10) Pub. No.: US 2021/0188760 A1
(43) Pub. Date: Jun. 24, 2021

(54) PROCESS FOR THE PURIFICATION OF A PERFLUOROCARBON COMPOSITION

(71) Applicant: 3M INNOVATIVE PROPERTIES COMPANY, St. Paul, MN (US)

(72) Inventors: Rudolf J. Dams, Antwerp (BE); Rudy W. Van Campenhout, Hoboken (BE); Matthias Conradi, Hemsloh (DE)

(21) Appl. No.: 17/123,420

(22) Filed: Dec. 16, 2020

(30) Foreign Application Priority Data
Dec. 20, 2019 (EP) 19218706.0

(51) Int. Cl. C07C 209/84; C07C 211/15

(52) U.S. Cl. CPC C07C 209/84; C07C 211/15

(57) ABSTRACT

The present disclosure relates to a process for the purification of a perfluorocarbon composition from a mixture comprising the perfluorocarbon composition and at least one non-perfluorinated hydrofluorocarbon compound, wherein the process comprises the steps of:
A. providing a continuous reactor comprising at least one reaction channel and mixing means;
B. providing reactants and reagents comprising:
   a) a mixture comprising a perfluorocarbon composition and at least one non-perfluorinated hydrofluorocarbon compound;
   b) a basic compound; and
   c) optionally, a liquid medium; and
C. incorporating the reactants and reagents into the reaction channel of the continuous reactor, thereby forming a reaction product stream comprising the purified perfluorocarbon composition.

### TECHNICAL FIELD

The present disclosure relates generally to the manufacturing of perfluorocarbon compositions. More specifically, the disclosure relates to a process for the purification of a perfluorocarbon composition from a mixture comprising the perfluorocarbon composition and at least one non-perfluorinated hydrofluorocarbon compound.

### BACKGROUND

Perfluorocarbon compositions, also referred to as PFC, have been known for decades and used in various applications due in particular to their chemical inertness and advantageous physical properties. Perfluorocarbons are non-conductive, thermally and chemically stable and are therefore ideal for single phase heat transfer fluid applications, especially in the electronics and semiconductor industry, and for fire extinction. Other known applications for such perfluorocarbons include use in cosmetics and in medical applications. Exemplary perfluorocarbon fluids are commercially available under the trade designation Fluorinert™ from the 3M Company.

Perfluorocarbon compounds may be prepared e.g. by direct fluorination of hydrocarbons or by electrochemical fluorination (ECF), also known as the Simons' process. The electrochemical fluorination process, which involves electrolysis of a hydrocarbon dissolved in hydrogen fluoride, is usually incomplete and produces for example non-perfluorinated derivatives, in particular non-perfluorinated hydrofluorocarbon compounds, as unwanted by-products. As these non-perfluorinated derivatives do not share the same advantageous properties as their perfluorinated counterparts, they typically have to be eliminated or extracted from the reaction mixture resulting from the electrochemical fluorination process.

One known method to chemically eliminate these unwanted derivatives (also referred to a stabilization) and thereby obtain purified perfluorocarbon compounds, is a batch process involving treating the reaction mixture under basic conditions, elevated temperature (typically greater than 100° C.), relatively low pressure (typically no greater than 1 MPa), and for a period of several hours (typically greater than 20 hours).

### SUMMARY

Without contesting the technical advantages associated with the processes known in the art, there is still a need for a convenient, stable, fast, efficient, and cost-effective process for the purification of a perfluorocarbon composition from a mixture comprising the perfluorocarbon composition and at least one non-perfluorinated hydrofluorocarbon compound. Other advantages of the process of the present disclosure will be apparent from the following disclosure.

According to one aspect, the present disclosure relates to a process for the purification of a perfluorocarbon composition from a mixture comprising the perfluorocarbon composition and at least one non-perfluorinated hydrofluorocarbon compound, wherein the process comprises the steps of:
A. providing a continuous reactor comprising at least one reaction channel and mixing means;
B. providing reactants and reagents comprising:
   a) a mixture comprising a perfluorocarbon composition and at least one non-perfluorinated hydrofluorocarbon compound;
   b) a basic compound; and
   c) optionally, a liquid medium; and
C. incorporating the reactants and reagents into the reaction channel of the continuous reactor, thereby forming a reaction product stream comprising the purified perfluorocarbon composition.

In another aspect, the present disclosure is directed to a purified perfluorocarbon composition which is obtained from the process as described above.

According to still another aspect, the present disclosure relates to the use of a continuous reactor comprising at least one reaction channel and mixing means, for the purification of a perfluorocarbon composition.

### BRIEF DESCRIPTION OF THE DRAWINGS

FIG. 1 illustrates a schematic view of an exemplary apparatus suitable for carrying out the process according to the present disclosure.

FIG. 2 illustrates a schematic cross-sectional view of an exemplary continuous reactor for use in the process according to the present disclosure.

FIG. 3 illustrates a schematic top view of an exemplary mixing means for use in the process according to the present disclosure.

### DETAILED DESCRIPTION

According to one aspect, the present disclosure relates to a (continuous) process for the purification of a (saturated) perfluorocarbon composition from a (liquid) mixture comprising the (saturated) perfluorocarbon composition and at least one non-perfluorinated hydrofluorocarbon compound, wherein the process comprises the steps of:
A. providing a continuous reactor comprising at least one reaction channel and mixing means;
B. providing reactants and reagents comprising:
   a) a (liquid) mixture comprising a (saturated) perfluorocarbon composition and at least one non-perfluorinated hydrofluorocarbon compound;
   b) a basic compound; and
   c) optionally, a liquid medium; and
C. incorporating the reactants and reagents into the reaction channel of the continuous reactor, thereby forming a reaction product stream comprising the purified (saturated) perfluorocarbon composition.

In the context of the present disclosure, it has been surprisingly found that a process as described above provides a convenient, stable, safe, fast, efficient, versatile and cost-effective method for the purification of a perfluorocarbon composition from a mixture comprising the perfluorocarbon composition and at least one non-perfluorinated hydrofluorocarbon compound. It has been further surprisingly found that a continuous reactor as described above is particularly suitable for the purification of a perfluorocarbon composition from a mixture comprising the perfluorocarbon composition and at least one non-perfluorinated hydrofluorocarbon compound.

Advantageously, the process as described above allows producing purified perfluorocarbon compositions comprising very low levels of non-perfluorinated hydrofluorocarbon compound(s), in particular comprising less than 100 ppm, less than 80 ppm, less than 60 ppm, less than 50 ppm, less than 40 ppm, less than 30 ppm, less than 20 ppm, or even less than 15 ppm, of the (sum of the) non-perfluorinated hydrofluorocarbon compound(s).

In still another advantageous aspect of the present disclosure, the process as described above allows achieving this relatively high level of purification within a relatively short processing time, typically in a range of a few minutes. In an exemplary aspect, the processing time is no greater than 1800 seconds, no greater than 1200 seconds, no greater than 900 seconds, no greater than 600 seconds, no greater than 360 seconds, no greater than 240 seconds, no greater than 180 seconds, or even no greater than 120 seconds.

Advantageously still, the process as described above is a robust and production-efficient process. In an advantageous aspect, the process of the present disclosure further provides excellent control of the reaction temperature profile (efficient thermal management), in particular through ensuring rapid, very intensive and intimate mixing, as well as efficient transport of the starting material and intermediate reaction mixtures during the purification reaction process. In another advantageous aspect, the process of the present disclosure further provides excellent control and management of the reaction pressure profile. As such, the process of the present disclosure allows using a broad scope of possible starting reactants and reagents for the purification of a perfluorocarbon composition.

In a further advantageous aspect, the process as described above provides high yields of purified perfluorocarbon compositions having excellent purity and quality due to the efficient neutralization, elimination or substantial reduction of unwanted side reaction products such as e.g. non-perfluorinated hydrofluorocarbon compounds or olefinically unsaturated perfluorocarbon compounds, which might derive from the manufacturing process of the perfluorocarbon composition, in particular from the electrochemical fluorination process of hydrocarbons.

In yet another advantageous aspect of the present disclosure, the purification process as described herein may be conducted in the presence of a liquid medium, in particular a polar solvent such as water, in order to provide a multi-phase reaction system (typically a two-phase reaction system). Such multi-phase systems may not only provide more efficient transport of the starting material into the continuous reactor, but also more efficient and intimate mixing of the reactants and reagents into the reaction channel(s) of the continuous reactor during the purification reaction process. Such multi-phase systems may also facilitate the separation of the reaction products or by-products present in the reaction product stream resulting from the purification reaction process.

Without wishing to be bound by theory, it is believed that these excellent properties and benefits are due in particular to the specific combination of the use of a continuous (flow) reactor comprising at least one reaction channel and mixing means and the use of the specific reactants and reagents as mentioned above.

According to another aspect, the present disclosure relates to a (continuous) process for removing non-perfluorinated hydrofluorocarbon compound(s) from a (liquid) mixture comprising a (saturated) perfluorocarbon composition and at least one non-perfluorinated hydrofluorocarbon compound, wherein the process comprises the steps of:
A. providing a continuous reactor comprising at least one reaction channel and mixing means;
B. providing reactants and reagents comprising:
   a) a (liquid) mixture comprising a (saturated) perfluorocarbon composition and at least one non-perfluorinated hydrofluorocarbon compound;
   b) a basic compound; and
   c) optionally, a liquid medium; and
C. incorporating the reactants and reagents into the reaction channel(s) of the continuous reactor under continuous mixing, thereby forming a reaction product stream comprising the purified (saturated) perfluorocarbon composition.

In the context of the present disclosure, the term “perfluorocarbon composition” is meant to designate a composition consisting of at least one perfluorocarbon compound, typically more than one perfluorocarbon compound. The term “perfluorocarbon compound” is meant to refer not only to perfluorocarbon compounds with the formula CₓF_{y}, but also to those perfluorocarbon compounds including heteroatoms, such as N, S or O, wherein all C—H covalent bonds have been replaced by C—F covalent bonds. The term “saturated perfluorocarbon compound” is meant to designate perfluorocarbon compounds which do not comprise any olefinic unsaturation. The term “non-perfluorinated hydrofluorocarbon compound” is meant to designate compounds similar to the perfluorocarbon compounds, with the exception that not all C—H covalent bonds have been replaced by C—F covalent bonds. Examples of non-perfluorinated hydrofluorocarbon compounds include, but are not limited to, non-perfluorinated (hydrogen-containing) hydride derivatives (e.g. monohydride derivatives and dihydride derivatives) of the perfluorocarbon compounds. The terms “alpha-hydride derivatives, beta-hydride derivatives and gamma-hydride derivatives of perfluorinated trialkyl amines” are meant to refer to non-perfluorinated (hydrogen-containing) hydride derivatives perfluorinated trialkyl amines, wherein at least one C—H covalent bond in respectively the alpha, beta or gamma position with respect to the heteroatom, more specifically the nitrogen atom, has not been replaced by a C—F covalent bond. The term “olefinically unsaturated perfluorocarbon compound” is meant to designate a perfluorocarbon compound which comprises at least one olefinic unsaturation. The term “liquid medium” is meant to designate a medium (e.g. a solvent) which is fully in liquid state at the processing temperature, and which may advantageously have solvation capabilities, in particular towards solutes in solid state.

In the context of the present disclosure still, the term “continuous reactor”, also sometimes referred to as flow reactor, is meant to designate a chemical reactor which carries reactants and reagents as a flowing stream, whereby the reactants and reagents are continuously fed into the reactor, and the targeted product(s) emerge as continuous stream.

The term “addition stream” is meant to refer to the reactants, solvents and reagents flowing from an entry location to the reaction channel(s) of the continuous reactor.

The term “reaction channel” is meant to refer to a region or area of the continuous reactor where separate incoming reactants and reagents or addition streams are combined and contact one another. The reactants and reagents or addition streams mix and chemically react with one another thereby forming a reaction product stream.

The term “residence time” is meant to refer to the period of time the reaction product stream remains in the reaction channel(s) of the continuous reactor from the moment the reactants and reagents or addition streams are incorporated and mixed into the reaction channel(s) of the continuous reactor until the moment the reaction product stream exits the last reaction channel.

Continuous reactors for use herein are not particularly limited. Any continuous reactor comprising at least one reaction channel and mixing means may be used in the context of the present disclosure. Suitable continuous reactors for use herein will be easily identified by those skilled in the art, in the light of the present description. Exemplary continuous reactors for use herein are described for example in US-A1-2009/0043122 (Azzawi et al.).

In a typical aspect of the disclosure, the continuous reactor for use herein is selected from the group consisting of tubular reactors, plug flow reactors, piston flow reactors, microreactors, mesoreactors, micromixers, and any combinations thereof. Suitable continuous reactors for use herein are commercially available, for example, under the trade designation Miprowa®, from Ehrfeld Mikrotechnik GmbH (Germany).

According to an advantageous aspect, the reaction channel(s) for use herein extend (substantially) over the entire length of the continuous reactor.

According to another advantageous aspect, the continuous reactor for use herein comprises a plurality of reaction channels extending (substantially) over the entire length of the continuous reactor and are in fluid communications with each other.

In a typical aspect of the disclosure, the continuous reactor for use herein comprises a series of from 2 to 500, from 2 to 400, from 2 to 300, from 2 to 200, from 2 to 100, from 2 to 50, from 2 to 25, or even from 2 to 15 reaction channels in fluid communications with each other.

The reaction channels for use herein may be advantageously selected from the group of (substantially) linear longitudinal channels, non-linear channels (e.g. S-shaped channels or zig-zag channels), split channels, recombined channels, and any combinations thereof.

In a beneficial aspect, the reaction channels for use herein have an overall shape selected from the group of square, rectangular, triangular, circular, oval, trapezoidal, and any combinations or mixtures thereof.

In a particularly beneficial aspect, the reaction channels for use herein have a (substantially) rectangular shape.

According to one exemplary aspect of the disclosure, the reaction channel(s) of the continuous reactor for use herein have a (combined) internal volume of no greater than 500 ml, no greater than 400 ml, no greater than 300 ml, no greater than 280 ml, no greater than 250 ml, no greater than 220 ml, no greater than 200 ml, no greater than 180 ml, no greater than 150 ml, or even no greater than 120 ml.

According to one exemplary aspect of the disclosure, the reaction channel(s) of the continuous reactor for use herein have a (combined) internal volume of no greater than 100 liters, no greater than 50 liters, no greater than 25 liters, no greater than 10 liters, no greater than 5 liters, or even no greater than 1 liter.

According to one typical aspect of the disclosure, the reaction channel(s) of the continuous reactor for use herein have a (combined) internal volume of greater than 10 ml, greater than 20 ml, greater than 50 ml, greater than 80 ml, greater than 100 ml, or even greater than 120 ml.

According to another typical aspect of the disclosure, the reaction channel(s) of the continuous reactor for use herein have a (combined) internal volume of greater than 200 ml, greater than 500 ml, greater than 1000 ml, greater than 2500 ml, or even greater than 5000 ml.

According to one particularly advantageous aspect, the reaction channel(s) of the continuous reactor have a (combined) internal volume in a range from 10 ml to 500 ml, from 10 ml to 400 ml, from 20 ml to 300 ml, from 20 ml to 250 ml, from 30 ml to 250 ml, from 30 ml to 200 ml, from 40 ml to 200 ml, from 40 ml to 180 ml, from 50 ml to 180 ml, from 50 ml to 150 ml, or even from 50 ml to 150 ml.

According to another particularly advantageous aspect, the reaction channel(s) of the continuous reactor have a (combined) internal volume in a range from 1 liter to 100 liters, from 5 liters to 80 ml, from 10 liters to 80 liters, from 30 liters to 80 liters, from 40 liters to 80 liters, or even from 50 liters to 80 liters.

In an advantageous aspect, the continuous reactor for use herein, in particular the reaction channel(s), is actively thermally controlled, in particular thermally heated, in particular with thermal oil.

Mixing means for use herein are not particularly limited. Any mixing means commonly known in the art may be used in the context of the present disclosure, provided it is adapted to the selected continuous reactor. Suitable mixing means for use herein will be easily identified by those skilled in the art, in the light of the present description.

In a typical aspect, the mixing means for use herein take the form of structured elements designed to create a turbulent flow in the reaction channel(s), in particular over (substantially) the entire length of the reaction channels, more in particular over (substantially) the entire length of the continuous reactor, whereby the reactants and reagents are intensively mixed with each other.

In a beneficial aspect, the mixing means for use herein take the form of turbulence generating elements. Exemplary turbulence generating elements for use herein are described for example in US-A1-2005/0189092 (Jahn et al.) or in WO 2019/129665A1 (Kroschel et al.).

According to a particularly beneficial aspect, the mixing means for use herein take the form of a deflector (or comb-like structure) comprising a spindle and a plurality of rods longitudinally extending from the spindle, wherein the plurality of rods and the spindle form a plane, wherein the rods are regularly spaced apart and arranged parallel to each other, and wherein the rods are in particular inclined (i.e. are non-perpendicular) relative to the axis formed by the spindle, so as to form an angle in a range from 10 to 85 degrees, from 20 to 80 degrees, from 30 to 60 degrees, or even from 40 to 50 degrees, relative to the axis formed by the spindle.

According to a more beneficial aspect, the mixing means for use herein take the form of a semi-fishbone baffle.

In a typical aspect of the disclosure, the mixing means are at least partially, in particular fully, (removably) inserted into at least one of the reaction channel(s). Advantageously, the mixing means are at least partially, in particular fully, (removably) inserted into all the reaction channel(s).

In an advantageous aspect, at least one of the reaction channel(s) comprise a plurality of mixing means, which are in superimposed and at least partially, in particular fully, inserted into the channel(s). More advantageously, all the reaction channel(s) comprise a plurality of superimposed mixing means as described above.

In an even more advantageously aspect, the mixing means for use herein take the form of superimposed deflectors as defined above, and wherein the respective angles formed by the plurality of rods relative to the corresponding spindles are all dissimilar when compared alternatively.

According to one particular aspect of the process of the present disclosure, the step of incorporating the reactants and reagents into the reaction channel(s) of the continuous reactor is performed such that the reactants and reagents are subjected to intense (and continuous) mixing, in particular intense turbulent mixing in the reaction channel(s), more in particular over (substantially) the entire length of the continuous reactor.

According to another particular aspect, the step of incorporating the reactants and reagents into the reaction channel(s) of the continuous reactor is performed such that the reactants and reagents form an emulsion before the (continuous) mixing step, in particular a stable emulsion, more in particular an emulsion stable for a period greater than 5 seconds at a temperature of (about) 23° C.

According to still another particular aspect, the process of the present disclosure comprises the steps of:
A. providing a first addition stream comprising the (saturated) perfluorocarbon composition and the at least one non-perfluorinated hydrofluorocarbon compound;
B. providing a second addition stream comprising the basic compound and the optional liquid medium; and
C. incorporating the first addition stream and the second addition stream into the reaction channel(s) of the continuous reactor, thereby forming a reaction product stream comprising the purified (saturated) perfluorocarbon composition.

According to yet another particular aspect of the disclosure, the process comprises the steps of:
A. providing a first addition stream comprising the (saturated) perfluorinated perfluorocarbon composition and the at least one non-perfluorinated hydrofluorocarbon compound;
B. providing a second addition stream comprising the basic compound;
C. providing a third addition stream comprising the optional liquid medium; and
D. incorporating the first addition stream, the second addition stream and the third addition stream into the reaction channel(s) of the continuous reactor, thereby forming a reaction product stream comprising the purified (saturated) perfluorocarbon composition.

In a typical of the process for the purification of a perfluorocarbon composition, the reactants and reagents, and in particular the first addition stream, the second addition stream and the optional third addition stream, are pre-mixed prior to incorporation into the reaction channel(s) of the continuous reactor, in particular pre-mixed at a temperature of (about) 23° C.

In another typical aspect of the process, the reactants and reagents, and in particular the first addition stream, the second addition stream and the optional third addition stream, are pre-mixed prior to incorporation into the reaction channel(s) of the continuous reactor, at a temperature greater than 23° C., greater than 40° C., greater than 50° C., or even greater than 70° C. The pre-mixing temperature is typically chosen to be lower than the processing temperature and is typically chosen such that all the reactants and reagents, or the addition streams are liquid at the pre-mixing temperature.

In still another typical aspect of the process, the reactants and reagents, and in particular the first addition stream, the second addition stream and the optional third addition stream, are incorporated simultaneously into the reaction channel(s) of the continuous reactor, in particular at a temperature of (about) 23° C.

In an alternative aspect of the process, the reactants and reagents, and in particular the first addition stream, the second addition stream and the optional third addition stream, are incorporated into the reaction channel(s) of the continuous reactor in successive steps, in particular at a temperature of (about) 23° C.

According to an advantageous aspect of the disclosure, the residence time of the reaction product stream comprising the purified (saturated) perfluorocarbon composition in the reaction channel(s) of the continuous reactor is no greater than 1800 seconds, no greater than 1500 seconds, no greater than 1200 seconds, no greater than 1000 seconds, no greater than 900 seconds, no greater than 720 seconds, no greater than 600 seconds, no greater than 480 seconds, no greater than 360 seconds, no greater than 300 seconds, no greater than 240 seconds, no greater than 180 seconds, or even no greater than 120 seconds.

Perfluorocarbon compositions for use herein are not particularly limited. Any perfluorocarbon compositions commonly known in the art may formally be used in the context of the present disclosure. Suitable perfluorocarbon compositions for use herein will be easily identified by those skilled in the art, in the light of the present description.

In one typical aspect of the disclosure, the perfluorocarbon composition for use in the present process is a saturated perfluorocarbon composition, which comprises in particular at least one saturated perfluorocarbon compound.

According to one exemplary aspect, the perfluorocarbon composition for use herein comprises at least one perfluorocarbon compound selected from the group consisting of perfluorinated alkyls, perfluorinated ethers, perfluorinated amines, perfluorinated ketones, perfluorinated carboxylic acids, perfluorinated sulfonic acids, perfluorinated alkyl halides, and any isomers, combinations or mixtures thereof.

According to one advantageous aspect, the perfluorocarbon composition for use herein comprises at least one perfluorocarbon compound selected from the group consisting of perfluorinated alkyls, perfluorinated ethers, perfluorinated amines, and any isomers, combinations or mixtures thereof.

According to a more advantageous aspect, the perfluorocarbon composition for use herein comprises at least one perfluorocarbon compound selected from the group consisting of perfluorinated amines, in particular perfluorinated trialkyl amines, and any isomers, combinations or mixtures thereof.

According to an even more advantageous aspect, the perfluorocarbon composition for use herein comprises at least one perfluorocarbon compound which is a perfluorinated trialkyl amine selected in particular from the group consisting of perfluorinated triethylamine, perfluorinated tripropylamine, perfluorinated dipropylethylamine, perfluorinated propyldiethylamine, perfluorinated tributylamine, perfluorinated dibutylpropylamine, perfluorinated dibutylethylamine, perfluorinated butyl dipropylamine, perfluorinated tripentylamine, perfluorinated trihexylamine, perfluorinated triheptylamine, and any isomers, combinations or mixtures thereof. According to a particularly advantageous aspect of the disclosure, the perfluorocarbon composition for use herein comprises at least one perfluorocarbon compound selected from the group consisting of perfluorinated tripropylamine, perfluorinated tributylamine, perfluorinated tripentylamine, and any isomers, combinations or mixtures thereof.

According to a preferred aspect of the disclosure, the perfluorocarbon composition for use herein comprises at least one perfluorocarbon compound selected from the group consisting of perfluorinated tripropylamine, perfluorinated tributylamine, and any isomers, combinations or mixtures thereof.

Non-perfluorinated hydrofluorocarbon compounds for use herein are not particularly limited. Any non-perfluorinated hydrofluorocarbon compounds commonly known in the art may formally be used in the context of the present disclosure. Suitable non-perfluorinated hydrofluorocarbon compounds for use herein will be easily identified by those skilled in the art, in the light of the present description.

In one typical aspect of the disclosure, the non-perfluorinated hydrofluorocarbon compound for use in the present process is a non-perfluorinated (chemical) derivative of the perfluorocarbon composition, in particular from the perfluorocarbon compound. Typically, the non-perfluorinated hydrofluorocarbon compound(s) for use herein have a chemical structure resembling that of the perfluorocarbon compound. These resembling chemical structures, when compared to the perfluorocarbon compounds, typically contain undesired chemical moieties or atoms such as e.g. hydrogen atoms, olefinic unsaturations, or sub-structures deriving from side-reactions such as e.g. chain rearrangements, chain shortenings, chain breakings or chain extensions.

In another typical aspect of the disclosure, the non-perfluorinated hydrofluorocarbon compound for use in the present process is a non-perfluorinated (chemical) derivative of a hydrocarbon being subject to incomplete fluorination, such as e.g. electrochemical fluorination (ECF).

In another typical aspect, the non-perfluorinated hydrofluorocarbon compound for use in the present disclosure is a hydrogen-containing non-perfluorinated derivative, in particular a hydrogen-containing non-perfluorinated derivative of the at least one perfluorocarbon compound. In still another typical aspect, the non-perfluorinated hydrofluorocarbon compound for use in the present disclosure is a hydrogen-containing non-perfluorinated derivative of a hydrocarbon being subject to incomplete fluorination, such as e.g. electrochemical fluorination (ECF).

According to one exemplary aspect, the non-perfluorinated hydrofluorocarbon compound is selected from the group consisting of non-perfluorinated (hydrogen-containing) derivatives of perfluorinated alkyls, perfluorinated ethers, perfluorinated amines, perfluorinated ketones, perfluorinated carboxylic acids, perfluorinated sulfonic acids, perfluorinated alkyl halides, and any isomers, combinations or mixtures thereof.

According to one advantageous aspect, the non-perfluorinated hydrofluorocarbon compound for use herein is selected from the group consisting of non-perfluorinated (hydrogen-containing) derivatives of perfluorinated alkyls, perfluorinated ethers, perfluorinated amines, and any isomers, combinations or mixtures thereof.

According to another advantageous aspect, the non-perfluorinated hydrofluorocarbon compound for use herein is selected from the group consisting of non-perfluorinated (hydrogen-containing) derivatives of perfluorinated amines, in particular perfluorinated trialkyl amines, and any isomers, combinations or mixtures thereof.

According to a more advantageous aspect of the disclosure, the non-perfluorinated hydrofluorocarbon compound for use herein is a non-perfluorinated (hydrogen-containing) derivative of a perfluorinated trialkyl amine selected in particular from the group consisting of perfluorinated triethylamine, perfluorinated tripropylamine, perfluorinated dipropylethylamine, perfluorinated propyldiethylamine, perfluorinated tributylamine, perfluorinated dibutylpropylamine, perfluorinated dibutylethylamine, perfluorinated butyl dipropylamine, perfluorinated tripentylamine, perfluorinated trihexylamine, perfluorinated triheptylamine, and any isomers, combinations or mixtures thereof.

According to an even more advantageous aspect, the non-perfluorinated hydrofluorocarbon compound for use herein is selected from the group consisting of non-perfluorinated (hydrogen-containing) derivatives of perfluorinated tripropylamine, perfluorinated tributylamine, perfluorinated tripentylamine, and any isomers, combinations or mixtures thereof.

According to a preferred aspect, the non-perfluorinated hydrofluorocarbon compound for use herein is selected from the group consisting of non-perfluorinated (hydrogen-containing) derivatives of perfluorinated tripropylamine, perfluorinated tributylamine, perfluorinated tripentylamine, and any isomers, combinations or mixtures thereof.

According to a particularly preferred aspect, the non-perfluorinated hydrofluorocarbon compound for use herein is selected from the group consisting of non-perfluorinated (hydrogen-containing) derivatives of perfluorinated tripropylamine, perfluorinated tributylamine, and any isomers, combinations or mixtures thereof.

In another advantageous aspect, the non-perfluorinated hydrofluorocarbon compound for use herein is selected from the group consisting of non-perfluorinated (hydrogen-containing) hydride derivatives of the perfluorocarbon compound, and any isomers, combinations or mixtures thereof.

In still another advantageous aspect, the non-perfluorinated hydride derivatives of the perfluorocarbon compound for use herein are selected from the group consisting of monohydride derivatives, dihydride derivatives, trihydride derivatives, tetrahydride derivatives of the perfluorocarbon compound, and any isomers, combinations or mixtures thereof.

In still another advantageous aspect, the non-perfluorinated hydride derivatives of the perfluorocarbon compound for use herein are selected from the group consisting of monohydride derivatives, dihydride derivatives of the perfluorocarbon compound, and any isomers, combinations or mixtures thereof.

In still another advantageous aspect, the non-perfluorinated hydrofluorocarbon compound for use herein is selected from the group consisting of non-perfluorinated (hydrogen-containing) monohydride derivatives and dihydride derivatives of perfluorinated amines, in particular perfluorinated trialkyl amines, and any isomers, combinations or mixtures thereof.

In still another advantageous aspect, the non-perfluorinated hydrofluorocarbon compound for use herein is selected from the group consisting of non-perfluorinated (hydrogen-containing) monohydride derivatives and dihydride derivatives of perfluorinated trialkyl amines, and any isomers, combinations or mixtures thereof.

In still another advantageous aspect, the non-perfluorinated hydrofluorocarbon compound for use herein is selected from the group consisting of non-perfluorinated (hydrogen-containing) monohydride derivatives and dihydride derivatives of perfluorinated tripropylamine, perfluorinated tributylamine, perfluorinated tripentylamine, and any combinations or mixtures thereof.

In still another advantageous aspect, the non-perfluorinated hydrofluorocarbon compound for use herein is selected from the group consisting of non-perfluorinated (hydrogen-containing) monohydride derivatives and dihydride derivatives of perfluorinated tripropylamine, perfluorinated tributylamine, and any isomers, combinations or mixtures thereof.

In still another advantageous aspect, the non-perfluorinated hydrofluorocarbon compound for use herein is selected from the group consisting of non-perfluorinated (hydrogen-containing) mono-alpha-hydride derivatives, mono-beta-hydride derivatives, mono-gamma-hydride derivatives, di-alpha-hydride derivatives, di-beta-hydride derivatives, di-gamma-hydride derivatives of perfluorinated tripropylamine, perfluorinated tributylamine, and any isomers, combinations or mixtures thereof.

In still another advantageous aspect, the non-perfluorinated hydrofluorocarbon compound for use herein is selected from the group consisting of non-perfluorinated (hydrogen-containing) mono-alpha-hydride derivatives and di-alpha-hydride derivatives of perfluorinated tripropylamine, perfluorinated tributylamine, and any isomers, combinations or mixtures thereof.

Basic compounds for use herein are not particularly limited. Any basic compounds commonly known in the art may formally be used in the context of the present disclosure. Suitable basic compounds for use herein will be easily identified by those skilled in the art, in the light of the present description.

In one typical aspect, the basic compound for use herein is selected from the group consisting of organic bases, inorganic bases, and any combinations or mixtures thereof.

In another typical aspect, the basic compound for use herein is a nucleophilic basic compound, in particular a basic compound capable of nucleophilic attack towards olefinic unsaturations.

According to one advantageous aspect, the basic compound for use in the present disclosure has a pKa in water greater than 8.0, greater than 8.5, greater than 9, greater than 9.5, greater than 10, greater than 10.5, greater than 11, greater than 11.5, greater than 12, greater than 12.5, greater than 13, or even greater than 13.5.

According to another advantageous aspect, the basic compound for use herein is an inorganic base selected from the group consisting of alkali- or alkali earth metal hydroxides, and any mixtures thereof.

According to still another advantageous aspect, the basic compound for use herein is an alkali metal hydroxide selected from the group consisting of sodium hydroxide, potassium hydroxide, lithium hydroxide, and any mixtures thereof.

According to a preferred aspect, the basic compound for use herein is (or comprises) sodium hydroxide or potassium hydroxide, in particular potassium hydroxide.

According to an alternatively advantageous aspect of the disclosure, the basic compound for use herein is an organic base selected from the group consisting of primary amines, secondary amines, imines, amidines, and any combinations or mixtures thereof.

According to yet another advantageous aspect, the basic compound for use herein is selected from the group consisting of polyalkylene polyamines, in particular tetraalkylene pentamine.

According to a particularly advantageous aspect, the basic compound for use herein is a tetraalkylene pentaamine selected from the group consisting of tetraethylenepentamine, tetrapropylenepentamine, and any mixtures thereof.

According to a preferred aspect, the basic compound for use herein is selected to be (or comprise) tetraethylenepentamine.

According to another particularly advantageous aspect, the basic compound for use herein is selected from the group consisting of amidines, in particular cyclic amidines, more in particular bicyclic amidines.

According to another particularly advantageous aspect, the basic compound for use herein is a bicyclic amidine selected from the group consisting of 1,8-Diazabicyclo [5.4.0] undéc-7-ene, 1,5-Diazabicyclo [4.3.0] non-5-ene, and any mixtures thereof.

According to another preferred aspect, the basic compound for use herein is selected to be or comprise 1,8-Diazabicyclo [5.4.0] undec-7-ene.

Liquid mediums for use herein are not particularly limited. Any liquid mediums commonly known in the art may formally be used in the context of the present disclosure. Suitable liquid mediums for use herein will be easily identified by those skilled in the art, in the light of the present description.

Suitable liquid mediums for use herein shall be liquid at the processing temperature, but they may be solid at room temperature (i.e. about 23° C.). Solid mediums may be liquefied by e.g. thermal melting prior to incorporation into the continuous reactor.

In a typical aspect, the liquid medium for use herein is selected from the group consisting of polar solvents, non-polar solvents, and any combinations or mixtures thereof.

In one beneficial aspect of the disclosure, the liquid medium for use herein is a polar solvent selected from the group consisting of water, alcohols, ketones, amides, sulfoxides, and any combinations or mixtures thereof.

In one particularly beneficial aspect of the disclosure, the liquid medium for use herein is selected to be or comprise water.

In another particularly beneficial aspect of the disclosure, the liquid medium for use herein is a mixture of water and at least one alcohol. Advantageously, the alcohol for use as liquid medium has a boiling point greater than 50° C., greater than 80° C., greater than 100° C., or even greater than 120° C.

In another advantageous aspect, the liquid medium comprises an organic solvent, stable to basic conditions, such as certain alcohols, like methanol or tertiary butanol, or certain ketones, like di-isobutylketone

In another beneficial aspect of the disclosure, the liquid medium for use herein is a non-polar solvent selected from the group consisting of hydrocarbons, ethers, dioxanes, and any combinations or mixtures thereof.

According to one advantageous aspect of the process of the disclosure, the basic compound for use herein is an alkali metal hydroxide selected from the group consisting of sodium hydroxide and potassium hydroxide, and any mixtures thereof and wherein the alkali metal hydroxide is in solution in the liquid medium at a concentration greater than 10% wt./vol, greater than 20% wt./vol, greater than 30% wt./vol, greater than 35% wt./vol, greater than 40% wt./vol, greater than 45% wt./vol, greater than 50% wt./vol, greater than 55% wt./vol, greater than 60% wt./vol, or even greater than 65% wt./vol.

According to another advantageous aspect of the disclosure, the basic compound for use herein is an alkali metal hydroxide selected from the group consisting of sodium hydroxide and potassium hydroxide, and any mixtures thereof and wherein the alkali metal hydroxide is in solution in the liquid medium at a concentration no greater than 70% wt./vol, no greater than 65% wt./vol, or even no greater than 60% wt./vol.

According to another advantageous aspect of the disclosure, the basic compound for use herein is an alkali metal hydroxide selected from the group consisting of sodium hydroxide and potassium hydroxide, and any mixtures thereof and wherein the alkali metal hydroxide is in solution in the liquid medium at a concentration in a range from 20% wt./vol to 70% wt./vol, from 20% wt./vol to 65% wt./vol, from 30% wt./vol to 65% wt./vol, from 40% wt./vol to 65% wt./vol, from 40% wt./vol to 60% wt./vol, or even from 40% wt./vol to 55% wt./vol.

According to another advantageous aspect of the disclosure, the basic compound for use herein is an inorganic base, wherein the vol/vol ratio of [the inorganic base] to [the mixture of the perfluorocarbon composition and the at least one non-perfluorinated hydrofluorocarbon compound] is at least 1/3, at least 1/2, at least 1/1, at least 2/1, at least 3/1, at least 4/1, at least 5/1, at least 6/1, at least 7/1, at least 8/1, at least 9/1, or even at least 10/1.

According to still another advantageous aspect of the disclosure, the basic compound for use herein is an inorganic base, and wherein the vol/vol ratio of [the inorganic base] to [the mixture of the perfluorocarbon composition and the at least one non-perfluorinated hydrofluorocarbon compound] is in a range from 1/3 to 10/1, from 1/2 to 10/1, from 1/1 to 10/1, from 1/1 to 8/1, from 1/1 to 6/1, from 2/1 to 5/1, from 3/1 to 5/1, or even from 4/1 to 5/1.

According to still another advantageous aspect of the disclosure, the basic compound for use herein is an organic base, and wherein the vol/vol ratio of [the organic base] to [the mixture of the perfluorocarbon composition and the at least one non-perfluorinated hydrofluorocarbon compound] is no greater than 5/1, no greater than 4/1, no greater than 3/1, no greater than 2/1, or even no greater than 1/1.

According to still another advantageous aspect of the disclosure, the basic compound for use herein is an organic base, wherein the reactants and reagents further comprise a liquid medium, and wherein the vol/vol ratio of [the organic base] to [the liquid medium] is at least 1/3, at least 1/2, at least 1/1, at least 2/1, at least 4/1, at least 6/1, at least 8/1, or even at least 10/1.

In one advantageous aspect of these specific processes, the organic base for use in these advantageous aspects is selected from the group consisting of polyalkylene polyamines and amidines.

In another advantageous aspect of these specific processes, the liquid medium for use in these advantageous aspects is selected from the group consisting of water, organic solvents, in particular alcohols, and any combinations or mixtures thereof.

In a more advantageous aspect of these specific processes, the liquid medium for use in these advantageous aspects is selected to be or comprise water.

According to still another advantageous aspect of the disclosure, the reaction product stream comprises less than 100 ppm, less than 80 ppm, less than 60 ppm, less than 50 ppm, less than 40 ppm, less than 30 ppm, less than 20 ppm, less than 15 ppm, or even less than 10 ppm, of the (sum of the) non-perfluorinated hydrofluorocarbon compound(s). Advantageously, these non-perfluorinated hydrofluorocarbon compounds are selected from the group consisting of monohydride derivatives, dihydride derivatives of the perfluorocarbon compound, and any isomers, combinations or mixtures thereof.

In one exemplary aspect of the process according to the present disclosure, the (liquid) mixture for use herein further comprises olefinically unsaturated perfluorocarbon compounds.

In another exemplary aspect of the disclosure, the (liquid) (reaction) mixture for use herein comprises:
a) from 70 to 99 mol % of the (saturated) perfluorocarbon composition;
b) from 1 to 25 mol % of the (sum of the) non-perfluorinated hydrofluorocarbon compound(s); and
c) optionally, from 0 to 5 mol % of the (sum of the) olefinically unsaturated perfluorocarbon compounds;
   wherein the mol % are based on the total number of moles of fluorocarbon compounds present in the (liquid) (reaction) mixture.

In still another exemplary aspect of the disclosure, the (liquid) (reaction) mixture for use herein is obtained from a direct fluorination process of hydrocarbons or from an electrochemical fluorination (ECF) process of hydrocarbons.

In one particular aspect of the disclosure, the (liquid) (reaction) mixture for use herein is obtained from an electrochemical fluorination (ECF) process of hydrocarbons.

According to an advantageous aspect of the process of the disclosure, the temperature of the reaction channel(s) of the continuous reactor is greater than 100° C., greater than 120° C., greater than 150° C., greater than 180° C., greater than 200° C., greater than 220° C., or even greater than 250° C.

According to another advantageous aspect of the process, the temperature of the reaction channel(s) of the continuous reactor is in a range from 100° C. to 300° C., from 120° C. to 300° C., from 150° C. to 300° C., from 150° C. to 280° C., from 180° C. to 270° C., from 190° C. to 270° C., from 200° C. to 270° C., from 220° C. to 270° C., from 230° C. to 270° C., from 240° C. to 270° C., or even from 250° C. to 270° C.

According to still another advantageous aspect of the process, the pressure of the reaction channel(s) of the continuous reactor is greater than 0.5 MPa, greater than 1 MPa, greater than 1.5 MPa, greater than 2.0 MPa, greater than 2.5 MPa, greater than 3.0 MPa, greater than 3.5 MPa, or even greater than 4.0 MPa.

According to yet another advantageous aspect of the process, the pressure of the reaction channel(s) of the continuous reactor is in a range from 0.6 MPa to 6.0 MPa, from 0.8 MPa to 6.0 MPa, from 1.0 MPa to 6.0 MPa, from 1.0 MPa to 5.5 MPa, from 1.0 MPa to 5.0 MPa, from 1.5 MPa to 5.0 MPa, from 2.0 MPa to 5.0 MPa, from 2.0 MPa to 4.5 MPa, from 2.5 MPa to 4.5 MPa, from 2.5 MPa to 4.0 MPa, or even from 3.0 MPa to 4.0 MPa.

According to an advantageous aspect of the process of the disclosure, the continuous reactor for use herein is part of an apparatus which further comprises a cooling equipment which is arranged (directly) downstream of the continuous reactor.

Cooling equipment for use herein are not particularly limited. Any cooling equipment commonly known in the art may formally be used in the context of the present disclosure. Suitable cooling equipment for use herein will be easily identified by those skilled in the art, in the light of the present description.

In an advantageous aspect of the disclosure, the cooling equipment for use herein is selected from the group consisting of (coaxial) heat-exchangers, meander reactors, and any combinations thereof.

In a typical aspect of the disclosure, the cooling equipment for use herein is actively thermally controlled, in particular thermally cooled, in particular with water.

In an exemplary aspect, the cooling equipment for use in the present disclosure is designed such as to provide the reaction product stream comprising the purified perfluorocarbon composition and exiting the continuous reactor with a temperature no greater than 100° C., no greater than 90° C., no greater than 80° C., no greater than 70° C., no greater than 60° C., or even no greater than 50° C.

In another exemplary aspect, the cooling equipment for use in the present disclosure is designed such as to provide the reaction product stream exiting the continuous reactor with a temperature in a range from 20° C. to 100° C., from 30° C. to 100° C., from 30° C. to 100° C., from 40° C. to 100° C., from 45° C. to 100° C., from 50° C. to 100° C., from 55° C. to 100° C., from 55° C. to 90° C., from 60° C. to 90° C., or even from 60° C. to 80° C.

According to an advantageous aspect of the process of the disclosure, the apparatus for use herein further comprises a (back-)pressure regulation equipment which is in particular arranged (directly) downstream of the cooling equipment.

Pressure regulation equipment for use herein are not particularly limited. Any pressure regulation equipment commonly known in the art may formally be used in the context of the present disclosure. Suitable pressure regulation equipment for use herein will be easily identified by those skilled in the art, in the light of the present description.

In an exemplary aspect, the (back-)pressure regulation equipment is selected from the group consisting of back-pressure spring-loaded regulators, back-pressure dome-loaded regulators, and any combinations thereof.

According to another advantageous aspect of the process of the disclosure, the apparatus for use herein further comprises a pre-mixing equipment, which is arranged (directly) upstream of the continuous reactor.

Pre-mixing equipment for use herein are not particularly limited. Any pre-mixing equipment commonly known in the art may formally be used in the context of the present disclosure. Suitable pre-mixing equipment for use herein will be easily identified by those skilled in the art, in the light of the present description.

In an exemplary aspect, the pre-mixing equipment for use herein is selected from the group consisting of capillary mixers, and any combinations thereof.

Advantageously, the pre-mixing equipment for use herein is coupled with at least one pump which is meant to assist incorporating reactants and/or reagents into the reaction channel(s) of the continuous reactor.

According to a beneficial aspect, the pump for use in combination with the pre-mixing equipment is selected from the group consisting of gear pumps, syringe pumps, HPLC pumps, piston pumps, and any combinations thereof.

Advantageously, the pump for use herein is controlled with a flow meter, in particular a mass flow meter.

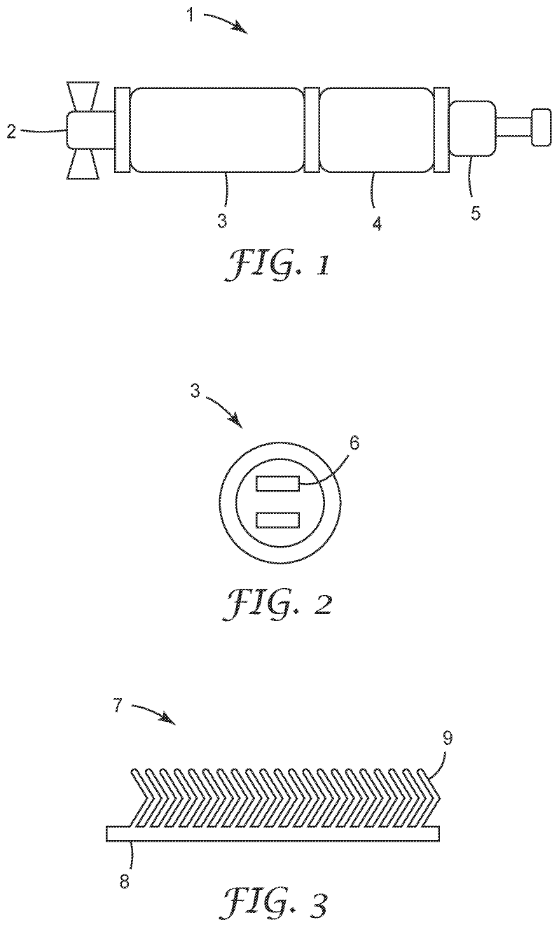

FIG. 1 illustrates a schematic view of an exemplary apparatus suitable for carrying out the process according to the present disclosure, wherein the apparatus 1 comprises a pre-mixing equipment 2, a continuous reactor 3 arranged directly downstream of the pre-mixing equipment 2, a cooling equipment 4 which is arranged directly downstream of the continuous reactor 3, and wherein the apparatus 1 further comprises a back-pressure regulation equipment 5 arranged directly downstream of the cooling equipment 4.

FIG. 2 illustrates a schematic cross-sectional view of an exemplary continuous reactor 3 for use in the process according to the present disclosure, wherein the continuous reactor 3 is shown as comprising two reaction channels 6.

FIG. 3 illustrates a schematic top view of an exemplary mixing means 7 for use in the process according to the present disclosure, wherein the mixing means 7 is in the form of a deflector comprising a spindle 8 and a plurality of rods 9 longitudinally extending from the spindle 8.

According to another aspect, the present disclosure relates to a purified perfluorocarbon composition obtained from the process as described above.

In an advantageous aspect, the purified perfluorocarbon composition according to the present disclosure comprises less than 100 ppm, less than 80 ppm, less than 60 ppm, less than 50 ppm, less than 40 ppm, less than 30 ppm, less than 20 ppm, or even less than 10 ppm, of the (sum of the) non-perfluorinated hydrofluorocarbon compound(s).

In another advantageous aspect, the purified perfluorocarbon composition according to the present disclosure comprises less than 100 ppm, less than 80 ppm, less than 60 ppm, less than 50 ppm, less than 40 ppm, less than 30 ppm, less than 20 ppm, less than 15 ppm, or even less than 10 ppm, of the sum of the non-perfluorinated hydrofluorocarbon compound(s) and the olefinically unsaturated perfluorocarbon compound(s).

In a particular aspect of the purified perfluorocarbon composition, the non-perfluorinated hydrofluorocarbon compounds are selected from the group consisting of monohydride derivatives, dihydride derivatives of the perfluorocarbon compound, and any isomers, combinations or mixtures thereof.

According to still another aspect, the present disclosure relates to the use of a continuous (flow) reactor comprising at least one reaction channel and mixing means, for the purification of a (saturated) perfluorocarbon composition.

According to yet another aspect, the present disclosure relates to the use of continuous (flow) reactor comprising at least one reaction channel and mixing means, for the (chemical) removal of non-perfluorinated hydrofluorocarbon compounds.

According to yet another aspect, the present disclosure relates to the use of a continuous (flow) reactor comprising at least one reaction channel and mixing means, for the (chemical) removal of olefinically unsaturated perfluorocarbon compounds.

According to yet another aspect, the present disclosure relates to the use of an apparatus as described above for the purification of a (saturated) perfluorocarbon composition.

According to yet another aspect, the present disclosure relates to the use of an apparatus as described above for the (chemical) removal of non-perfluorinated hydrofluorocarbon compounds.

According to yet another aspect, the present disclosure relates to the use of an apparatus as described above for the (chemical) removal of olefinically unsaturated perfluorocarbon compounds.

### EXAMPLES

The present disclosure is further illustrated by the following examples. These examples are merely for illustrative purposes only and are not meant to be limiting on the scope of the appended claims.

The following abbreviations are used in this section: NMR=nuclear magnetic resonance, ml=milliliters, min=minutes, ppm=parts per million. Abbreviations of materials used in this section, as well as descriptions of the materials, are provided in Table 1.

TABLE 1

| Material | Description |
|---|---|
| UPFC-1 | Unpurified perfluorocarbon composition comprising |
| | PTPA (perfluorotripropylamine,) which has a residual |
| | amount of non-perfluorinated hydrofluorocarbon |
| | (NPHFC) compounds of about 230 ppm |
| | as determined by Gas Chromatography, available |
| | from 3M Belgium, Zwijndrecht. Composition |
| | UPFC-1 is obtained from the electrochemical |
| | fluorination of hydrocarbon tripropylamine (TPA). |
| UPFC-2 | Unpurified perfluorocarbon composition comprising |
| | PTPA (perfluorotripropylamine,) which has a residual |
| | amount of non-perfluorinated hydrofluorocarbon |
| | (NPHFC) compounds of about 236 ppm |
| | as determined by Gas Chromatography, available |
| | from 3M Belgium, Zwijndrecht. Composition |
| | UPFC-2 is obtained from the electrochemical |
| | fluorination of hydrocarbon tripropylamine (TPA). |
| UPFC-3 | Unpurified perfluorocarbon composition comprising |
| | PTPA (perfluorotripropylamine,) which has a residual |
| | amount of non-perfluorinated hydrofluorocarbon |
| | (NPHFC) compounds of about 262 ppm |
| | as determined by Gas Chromatography, available |
| | from 3M Belgium, Zwijndrecht. Composition UPFC-3 |
| | is obtained from the electrochemical |
| | fluorination of hydrocarbon tripropylamine (TPA). |
| UPFC-4 | Unpurified perfluorocarbon composition comprising |
| | PTBA (perfluorotributylamine,) which has a residual |
| | amount of non-perfluorinated hydrofluorocarbon |
| | (NPHFC) compounds of about 288 ppm |
| | as determined by Gas Chromatography, available |
| | from 3M Belgium, Zwijndrecht. Composition |
| | UPFC-4 is obtained from the electrochemical |
| | fluorination of hydrocarbon tributylamine (TBA). |
| KOH | Potassium hydroxide in aqueous solution, available at |
| | various wt./vol grades from Aldrich, Belgium |
| TEPA | Tetraethylenepentamine, available from Aldrich, Belgium |
| DBU | 1,8-Diazabicyclo [5.4.0] undec-7-ene, available |
| | from Aldrich, Belgium |
| NPHFC | Non-perfluorinated hydrofluorocarbon (NPHFC) |
| | compounds, as obtained in the examples. |

Test Methods and Characterization:

Level of Residual NPHFC Compounds

The term “residual NPHFC level” is used throughout this section to designate the level of residual non-perfluorinated hydrofluorocarbon compounds (in ppm) present after the purification process. The level of residual NPHFC compounds is determined by ¹H-NMR and ¹⁹F NMR spectroscopy on the fluorine-containing phase of the reaction mixture which has been simply washed with deionized water and filtered over a 616 WA grade paper filter, as described below, under “Characterization.” Gas Chromatography (GC) is also used to assess the quality of the purification process.

Characterization

NMR: Analysis by NMR is made using a Bruker Avance 300 Digital NMR spectrometer equipped with Bruker 5 mm BBFO 300 MHz Z-gradient high resolution-ATM probe. The samples are placed in NMR tubes available under the trade designation “WG-SM-ECONOMY” from Aldrich, Belgium. CFC13 (trichlorofluoromethane, available from Aldrich, Belgium) is added as a zero-ppm reference. A capillary insert with acetone-D6 is added for shimming.

Fluorine and hydrogen NMR spectra are acquired using the following standard parameters:
Pulse Angle: 30°
Number of Scans: 256
Acquisition Time: 5.0 s

Except where noted, NMR confirmed the identity of the desired products.

Equipment Employed:

### Examples 1 to 5

The experiments and reactions are performed using the MMRS® system commercially available from Ehrfeld Mikrotechnik GmbH, Germany. The system consists of a capillary mixer with two Miprowa® continuous lab reactors mounted in series and having each an internal volume of 30 ml and eight reaction rectangular channels (3 mm×18 mm). Each reaction channel is provided with 3 layers of mixing means as depicted in WO 2019/129665A1 (Kroschel et al.) FIGS. 1 and 3. The system further comprises a coaxial tube-in-tube heat exchanger type 0309-4-0001-F (commercially available from Ehrfeld Mikrotechnik GmbH, Germany) as cooling equipment and a Swagelok back pressure regulator type 0613-1-02xy-T (commercially available from Ehrfeld Mikrotechnik GmbH, Germany) maintaining the pressure in the reactor channels between 1.5 MPa and 3.0 MPa. The continuous reactors and the heat exchanger are controlled separately via Lauda thermostats (thermal oil Fragoltherm in the reactors, and deionized water in the heat exchanger). The reactants and reagents are transported into the continuous reactor using a Coriolis flow meter (commercially available from Bronkhorst) controlled with micro annular gear pumps (commercially available from HNP Mikrosysteme). The automatization software package is LabVision available from HiTec Zang, Germany.

### Examples 6 to 11

The experiments and reactions are performed using the MMRS® system commercially available from Ehrfled Mikrotechnik GmbH. The system consists of a capillary mixer with one 1-meter long Miprowa® continuous lab reactor having an internal volume of 120 ml and three reaction rectangular channels (3 mm×18 mm). Each reaction channel is provided with 3 layers of mixing means as depicted in WO 2019/129665A1 (Kroschel et al.) FIGS. 1 and 3. The system further comprises a coaxial tube-in-tube heat exchanger type 0309-4-0001-F (commercially available from Ehrfeld Mikrotechnik GmbH, Germany) as cooling equipment and a Swagelok back pressure regulator type 0613-1-02xy-T (commercially available from Ehrfeld Mikrotechnik GmbH, Germany) maintaining the pressure in the reactor channels between 1.5 MPa and 3.0 MPa. The continuous reactors and the heat exchanger are controlled separately via Lauda thermostats (thermal oil Fragoltherm in the reactors, and deionized water in the heat exchanger). The reactants and reagents are transported into the continuous reactor using a Coriolis flow meter (commercially available from Bronkhorst) controlled with micro annular gear pumps (commercially available from HNP Mikrosysteme). The automatization software package is LabVision available from HiTec Zang.

### EXAMPLES

### Examples 1 to 3 and Comparative Example 1

For Ex.1 to Ex.3 and comparative example CE-1, the following general procedure is carried out using the processing conditions and parameters as described in Table 2 below. The basic compound (KOH solution or neat TEPA) and the unpurified perfluorocarbon composition UPFC-1 are simultaneously incorporated into the first continuous reactor in two separate addition streams. Comparative example CE-1 is the starting unpurified perfluorocarbon composition not being subject to the purification process. The vol/vol ratios of the various reactants and reagents, the reaction temperature and the residence time (RT in min), as well as the residual NPHFC level as determined by ¹H-NMR and ¹⁹F NMR spectroscopy, are specified in Table 2 below.

TABLE 2

| | | | | Temperature | | Residual |
|---|---|---|---|---|---|---|
| | Stream | Stream | Vol/vol ratio | reaction | RT | NPHFC |
| Example | I | II | (base/UPFC) | (° C.) | (min) | (ppm) |
| Ex. 1 | KOH | UPFC-1 | 5/1 | 250 | 6 | 99 |
| | (50%) | | | | | |
| Ex. 2 | TEPA | UPFC-1 | 5/1 | 250 | 3 | 30 |
| Ex. 3 | TEPA | UPFC-1 | 5/1 | 230 | 12 | 58 |
| CE-1 | — | UPFC-1 | — | — | — | 230 |

### Examples 4 to 5 and Comparative Example 1

For Ex.4 to Ex.5 and comparative example CE-1, the following general procedure is carried out using the processing conditions and parameters as described in Table 3 below. The basic compound (TEPA), the liquid medium (water) and the unpurified perfluorocarbon composition UPFC-1 are simultaneously incorporated into the first continuous reactor in three separate addition streams. Comparative example CE-1 is the starting unpurified perfluorocarbon composition not being subject to the purification process. Example 5 does not comprise a liquid medium. The vol/vol/vol ratios of the various reactants and reagents, the reaction temperature and the residence time (RT in min), as well as the residual NPHFC level as determined by ¹H-NMR and ¹⁹F NMR spectroscopy, are specified in Table 3 below.

TABLE 3

| | | | | | | | Residual |
|---|---|---|---|---|---|---|---|
| | Stream | Stream | Stream | Vol/vol/vol ratio | T | RT | NPHFC |
| Example | I | II | III | (base/UPFC/H₂O) | (° C.) | (min) | (ppm) |
| Ex. 4 | TEPA | UPFC-1 | H₂O | 1/1/0.1 | 250 | 18 | 41 |
| Ex. 5 | TEPA | UPFC-1 | — | 1/2/0 | 240 | 6 | 95 |
| CE-1 | — | UPFC-1 | — | — | — | — | 230 |

### Examples 6 to 9 and Comparative Examples 2 and 3

For Ex.6 to Ex.9 and comparative examples CE-2 and CE-3, the following general procedure is carried out using the processing conditions and parameters as described in Table 4 below. The basic compound (TEPA or DBU), the liquid medium (water) and the unpurified perfluorocarbon compositions (UPFC-2 or UPFC-4) are simultaneously incorporated into the first continuous reactor in three separate addition streams. Comparative examples CE-2 and CE-3 are the starting unpurified perfluorocarbon compositions not being subject to the purification process. The vol/vol/vol ratios of the various reactants and reagents, the reaction temperature and the residence time (RT in min), as well as the residual NPHFC level as determined by ¹H-NMR and ¹⁹F NMR spectroscopy, are specified in Table 4 below.

TABLE 4

| | | | | | | | Residual |
|---|---|---|---|---|---|---|---|
| | Stream | Stream | Stream | Vol/vol/vol ratio | T | RT | NPHFC |
| Example | I | II | III | (base/UPFC/H₂O) | (° C.) | (min) | (ppm) |
| Ex. 6 | TEPA | UPFC-2 | H₂O | 0.9/1/0.1 | 270 | 6 | 80 |
| Ex. 7 | DBU | UPFC-2 | H₂O | 0.9/1/0.1 | 270 | 6 | 24 |
| Ex. 8 | DBU | UPFC-2 | H₂O | 0.9/2/0.1 | 270 | 6 | 16 |
| Ex. 9 | DBU | UPFC-3 | H₂O | 0.9/1/0.1 | 270 | 6 | 18 |
| CE-2 | — | UPFC-2 | — | — | — | — | 236 |
| CE-3 | — | UPFC-4 | — | — | — | — | 288 |

### Examples 10 to 11 and Comparative Example 4

For Ex.10 to Ex.11 and comparative example CE-4, the following general procedure is carried out using the processing conditions and parameters as described in Table 5 below. The basic compound (KOH solution) and the unpurified perfluorocarbon composition UPFC-4 are simultaneously incorporated into the first continuous reactor in two separate addition streams. Comparative example CE-4 is the starting unpurified perfluorocarbon composition not being subject to the purification process. The vol/vol/ratios of the various reactants and reagents, the reaction temperature and the residence time (RT in min), as well as the residual NPHFC level as determined by ¹H-NMR and ¹⁹F NMR spectroscopy, are specified in Table 5 below.

TABLE 5

| | | | | Temperature | | Residual |
|---|---|---|---|---|---|---|
| | Stream | Stream | Vol/vol ratio | reaction | RT | NPHFC |
| Example | I | II | (base/UPFC) | (° C.) | (min) | (ppm) |
| Ex. 10 | KOH | UPFC-3 | 5/1 | 260 | 3 | 11 |
| | (50%) | | | | | |
| Ex. 11 | KOH | UPFC-3 | 3/1 | 260 | 12 | 12 |
| | (50%) | | | | | |
| CE-4 | — | UPFC-3 | — | — | — | 262 |

1. A process for the purification of a perfluorocarbon composition from a mixture comprising the perfluorocarbon composition and at least one non-perfluorinated hydrofluorocarbon compound, wherein the process comprises the steps of:
A. providing a continuous reactor comprising at least one reaction channel and mixing means;
B. providing reactants and reagents comprising:
a) a mixture comprising a perfluorocarbon composition and at least one non-perfluorinated hydrofluorocarbon compound;
b) a basic compound; and
c) optionally, a liquid medium; and
C. incorporating the reactants and reagents into the reaction channel(s) of the continuous reactor, thereby forming a reaction product stream comprising the purified perfluorocarbon composition.

2. The process according to claim 1, wherein the perfluorocarbon composition comprises at least one perfluorocarbon compound selected from the group consisting of perfluorinated alkyls, perfluorinated ethers, perfluorinated amines, perfluorinated ketones, perfluorinated carboxylic acids, perfluorinated sulfonic acids, perfluorinated alkyl halides, and any isomers, combinations or mixtures thereof.

3. The process according to claim 1, wherein the perfluorocarbon composition comprises at least one perfluorocarbon compound selected from the group consisting of perfluorinated amines, in particular perfluorinated trialkyl amines, and any isomers, combinations or mixtures thereof.

4. The process according to claim 1, wherein the non-perfluorinated hydrofluorocarbon compound is a non-perfluorinated derivative of the perfluorocarbon composition, in particular from the perfluorocarbon compound, more in particular a hydrogen-containing non-perfluorinated derivative of the at least one perfluorocarbon compound.

5. The process according to claim 1, wherein the non-perfluorinated hydrofluorocarbon compound is selected from the group consisting of non-perfluorinated derivatives of perfluorinated alkyls, perfluorinated ethers, perfluorinated amines, perfluorinated ketones, perfluorinated carboxylic acids, perfluorinated sulfonic acids, perfluorinated alkyl halides, and any isomers, combinations or mixtures thereof.

6. The process according to claim 1, wherein the non-perfluorinated hydrofluorocarbon compound is selected from the group consisting of non-perfluorinated hydride derivatives of the perfluorocarbon compound, and any isomers, combinations or mixtures thereof.

7. The process according to claim 1, wherein the basic compound is selected from the group consisting of organic bases, inorganic bases, and any combinations or mixtures thereof.

8. The process according to claim 1, wherein the basic compound has a pKa in water greater than 8.0, greater than 8.5, greater than 9, greater than 9.5, greater than 10, greater than 10.5, greater than 11, greater than 11.5, greater than 12, greater than 12.5, greater than 13, or even greater than 13.5.

9. The process according to claim 1, wherein the basic compound is an inorganic base selected from the group consisting of alkali- or alkali earth metal hydroxides, and any mixtures thereof.

10. The process according to claim 1, wherein the basic compound is an organic base selected from the group consisting of primary amines, secondary amines, imines, amidines, and any combinations or mixtures thereof.

11. The process according to claim 1, wherein the liquid medium is selected from the group consisting of polar solvents, non-polar solvents, and any combinations or mixtures thereof.

12. The process according to claim 1, wherein the reaction product stream comprises less than 100 ppm, less than 80 ppm, less than 60 ppm, less than 50 ppm, less than 40 ppm, less than 30 ppm, less than 20 ppm, less than 15 ppm, or even less than 10 ppm, of the non-perfluorinated hydrofluorocarbon compound(s).

13. The process according to claim 1, wherein the residence time of the reaction product stream comprising the purified perfluorocarbon composition in the reaction channel(s) of the continuous reactor is no greater than 1800 seconds, no greater than 1200 seconds, no greater than 900 seconds, no greater than 600 seconds, no greater than 360 seconds, no greater than 240 seconds, no greater than 180 seconds, or even no greater than 120 seconds.

14. A purified perfluorocarbon composition obtained from the process according to claim 1.

15. (canceled)
